(19) United States
(12) Plant Patent Application Publication
(10) Pub. No.: US 2014/0165247 P1
(43) Pub. Date: Jun. 12, 2014

(54) New Guinea Impatiens Plant Named 'SAKIMP023'

(50) ;

(71) Applicant: SAKATA SEED CORPORATION, Yokohama (JP)

(72) Inventors: Chihiro Sato, Nagano (JP); Shinji Minemura, Nagano (JP)

(73) Assignee: Sakata Seed Corporation, Yokohama (JP)

(21) Appl. No.: 13/694,534

(22) Filed: Dec. 11, 2012

(51) Int. Cl. A01H 5/00

(52) U.S. Cl. PLT/318.4

(57) ABSTRACT

A New Guinea Impatiens plant particularly distinguished by having a pink flower color, strong root system and a vigorous plant growth habit is disclosed.

### GENUS AND SPECIES

Impatiens hybrida hort

### VARIETY DENOMINATION

‘SAKIMP023’

### BACKGROUND OF THE NEW PLANT

The present invention comprises of a new and distinct cultivar of New Guinea Impatiens, botanically known as Impatiens hybrida hort, and referred to by the variety name ‘SAKIMP023’. ‘SAKIMP023’ originated from a hybridization between the female Impatiens ‘NF-571B1’, an unpatented proprietary Impatiens breeding line with a salmon flower color and the male Impatiens line ‘NG-424’, an unpatented proprietary Impatiens breeding line with a lilac flower color in Misato, Japan.

In December 2007, the female parent line ‘NF-571B1’and male parent line ‘NG-424’ were crossed and a population of F₁ plants was created. The F₁ plants were evaluated in Misato, Japan in an open field trial. The criteria for plant selection included a beautiful pink flower color, strong root system and a vigorous plant growth habit. At the completion of the trial, one single-plant selection was made based on the aforementioned criteria and vegetatively propagated. From May to August 2009, the selection was evaluated in an open field in Misato, Japan. The selection subsequently was named ‘SAKIMP023’ and found to have its unique characteristics reproduce true to type in successive generations of asexual vegetative propagation.

### SUMMARY

The following are the most outstanding and distinguishing characteristics of this new variety when grown under normal horticultural practices in Salinas, Calif.

1. Pink flower color;

2. Strong root system; and

3. A vigorous plant growth habit.

### DESCRIPTION OF THE PHOTOGRAPHS

This Impatiens plant is illustrated by the accompanying photographs which show the plant's overall plant habit including form, foliage, and flowers. The photographs are of a three-month-old plant grown in Salinas, Calif. under greenhouse conditions in the spring of 2012. The colors shown are as true as can be reasonably obtained by conventional photographic procedures.

FIG. 1 shows the overall plant habit of the plant grown in a pot.

FIG. 2 shows the mature inflorescence of the plant.

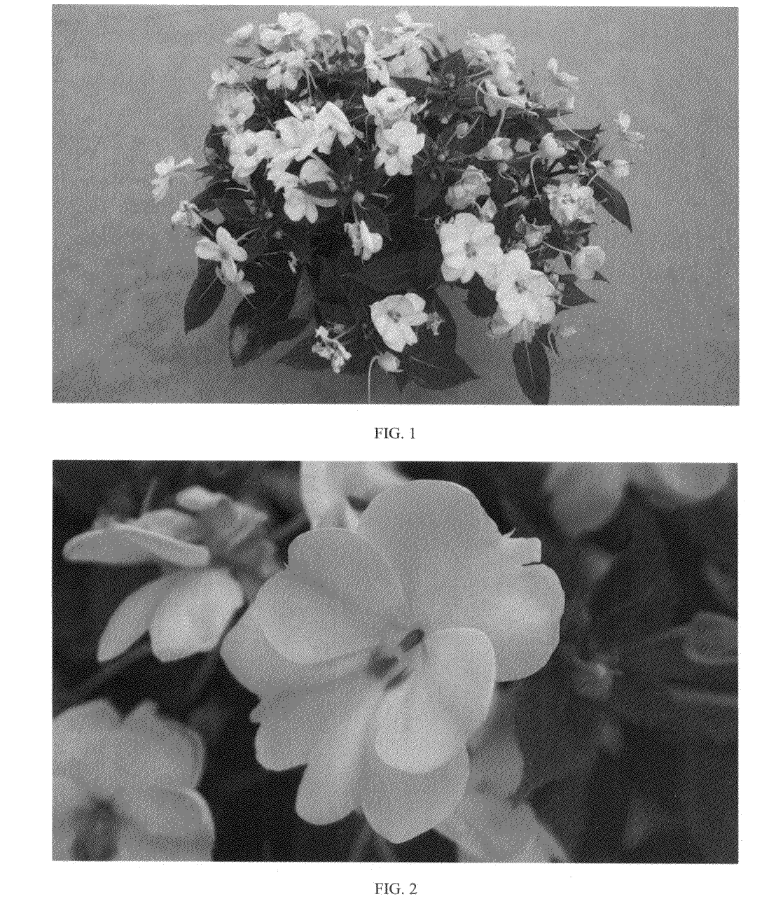

### DESCRIPTION OF THE NEW VARIETY

The following detailed descriptions set forth the distinctive characteristics of ‘SAKIMP023’. The data which define these characteristics were collected from asexual reproductions carried out in Salinas, Calif. Data was collected on three-month-old plants grown under greenhouse conditions in Salinas, Calif. in the summer of 2012. Color references are to The R.H.S. Colour Chart of The Royal Horticultural Society of London (R.H.S.), 4^{th} edition (2001).
Classification:
   Family.—Balsaminaceae.
   Botanical.—Impatiens interspecific cross (Impatiens hybrida hort).
   Common.—Impatiens
   Designation.—‘SAKIMP023’
Parentage:
   Female parent.—Proprietary Impatiens plant line ‘NF-571B1’ (unpatented).
   Male parent.—Proprietary Impatiens plant line ‘NG-424’ (unpatented).
Growth:
   Time to produce a rooted cutting.—4 weeks.
   Environmental conditions for plant growth.—The terminal 1.0 to 1.5 inches of an actively growing stem was excised. The vegetative cuttings were propagated in four weeks. The base of the cuttings were dipped for 1 to 2 seconds in a 1:9 solution of Dip 'N Grow (1 solution: 9 water) root inducing solution immediately prior to sticking into the cell trays. Cuttings were stuck into plastic cell trays having 98 cells, and containing a moistened peat moss-based growing medium. The cuttings were misted with water from overhead for 10 seconds every 30 minutes until sufficient roots were formed. Rooted cuttings were transplanted and grown in 6-inch plastic pots in a glass greenhouse located in Salinas, Calif. Pots contained a peat moss-based growing medium. Soluble fertilizer containing 20% nitrogen, 10% phosphorus and 20% potassium was applied once a day or every other day by overhead irrigation. Plants were fertilized every 2-3 days, 2 times in consecutive applications and then given one clear water application. Pots were top-dressed with a dry, slow release fertilizer containing 14% nitrogen, 14% phosphorus and 14% potassium. The typical average air temperature was 24° C.
   Time to bloom from propagation.—6 to 8 weeks.
Plant description:
   Habit.—Vigorous, large, well branching.
   Life cycle.—Tender perennial.
   Height (from soil line to top of foliage).—30.0 cm.
   Spread.—55.0 cm.
   Flowering requirements.—Will flower so long as temperature is above 5° C.
   Temperature tolerances.—Plants observed to continue flowering in a temperature range of 5° C. to 36° C. Plants can withstand high heat and humidity.
Branches:
   Number of branches.—1 main branch, 10 branches total.
   Length of branches.—Approximately 2.0 cm from soil line to first node, approximately 20.0 cm total.
   Diameter of branches.—2.0 cm, main branch.
   Color of branches.—RHS 143B (Green) and RHS 187A (Greyed-Purple).
Stems:
   General.—Strong; circular cross-section, smooth and shiny.
   Stem length.—11.0 cm.
   Diameter.—0.7 cm.
   Internode length.—5.0 cm.
   Color.—RHS 143B (Green) and RHS 187A (Greyed-Purple).
   Pubescence.—None.
   Anthocyanin color.—RHS 187A (Greyed-Purple).
Leaves:
   Arrangement.—Whorled with up to 5 leaves per node, opposite if only two leaves at one node.
   Shape.—Lanceolate, curled.
   Apex.—Acuminate.
   Base.—Attenuate.
   Margin.—Ciliate.
   Venation.—Pinnate.
   Venation color.—Upper vein color: RHS 145C (Yellow-Green) Lower vein color: RHS 145C (Yellow-Green).
   Variegation.—Absent.
   Surface appearance (both surfaces).—Dull, waxy.
   Surface pubescence (both surfaces).—Very slight.
   Surface pubescence color (both surfaces).—RHS N155A (White).
   Length.—10.0 cm.
   Width.—4.5 cm.
   Color.—Upper surface: Darker than RHS 147A (Yellow-Green) Lower surface: RHS 147B (Yellow-Green).
   Petiole.—Length: 1.5 cm Diameter: 0.5 cm Color: RHS 145C (Yellow-Green) with RHS 187C (Greyed-Purple) anthocyanin Texture: Smooth, glabrous.
   Fragrance.—Absent.
Flowers:
   Total number of flowers per node.—1 to 3 in bloom, about 4 to 6 flower buds per node.
   Total number of flower per plant.—Approximately 60 to 70 in bloom.
   Inflorescence type.—Single flower with spur.
   Lastingness of individual blooms on the plant.—14 days.
   Fragrance.—Absent.
Flower buds:
   Bud surface.—Glabrous.
   Bud length.—2.0 cm.
   Bud diameter.—1.5 cm.
   Bud shape.—Deltoid, longitudinal cross-section.
   Bud color.—RHS 65A (Red-Purple).
Peduncle:
   Peduncle length.—4.5 cm.
   Peduncle diameter.—0.2 cm.
   Peduncle color.—RHS 145B (Yellow-Green).
   Peduncle texture.—Smooth, glabrous.
Corolla:
   Shape.—Roughly circular with 5 radial petals.
   Diameter.—Approximately 6.5 cm.
   Depth.—2.0 cm.
   Petals.—Shape: Obcordate Apex: Emarginate (cleaved) Margin: Entire Base: Attenuate Pubescence: Glabrous Length: 2.5 cm Width: 4.2 cm Color: Upper: RHS 65B (Red-Purple) with RHS 65A (Red-Purple) at midvein Lower: RHS 65C (Red-Purple) with RHS 65B (Red-Purple) at midvein Eye zone: RHS N66A (Red-Purple) and RHS 145D (Yellow-Green).
Spur:
   Shape.—Tubular; curved downward.
   Length.—6.5 cm.
   Diameter.—0.2 cm.
   Color.—RHS 145D (Yellow-Green).
Calyx:
   Arrangement.—Composed of 2 sepals.
   Sepals.—Shape: Lanceolate Base: Subcordate Apex: Caudate Margin: Entire Texture: Glabrous Color: RHS 145B (Yellow-Green) and RHS 145C (Yellow-Green) and RHS 145A (Yellow-Green) at tip Length: 1.5 cm Diameter: 0.5 cm.
Reproductive organs:
   Stamen number.—Many.
   Stamen form.—Fused; split into 4 lobes.
   Filament length.—0.6 cm.
   Anther length.—0.6 cm.
   Stamen color.—Anther color: RHS 155A (White) Filament color: RHS 65A (Red-Purple).
   Pollen color.—RHS 155D (White).
   Pollen amount.—Abundant.
   Pollen description.—Powdery.
   Ovary.—Parietal.
   Ovary surface color.—RHS 145A (Yellow-Green).
   Pistil number.—5.
   Pistil length.—0.6 cm.
   Stigma color.—RHS 137A (Green).
   Style length.—0.7 cm.
   Style color.—RHS 137A (Green).
   Seed production.—Absent.
Disease and insect resistance: No particular resistance or susceptibility observed

### COMPARISON WITH PARENTAL LINES AND KNOWN VARIETY

‘SAKIMP023’ is a new and unique variety of Impatiens owing to its pink flower color, strong root system and a vigorous plant growth habit. ‘SAKIMP023’ is distinguished from its parents mainly by flower color as shown in Table 1 below:

TABLE 1
Comparison with Parental Lines

| | | Female Parent | Male Parent |
|---|---|---|---|
| Characteristic | ‘SAKIMP023’ | ‘NF-571B1’ | ‘NG-424’ |
| Flower color | Pink | Salmon | Lilac |

‘SAKIMP023’ is a new and unique variety of Impatiens owing to its pink flower color, strong root system and a vigorous plant growth habit. ‘SAKIMP023’ is most similar to the commercial Impatiens variety ‘SAKIMP013’ (U.S. Plant Pat. No. 19,597), commercially known as SunPatiens® ‘Compact Blush Pink’; however there are differences in the flower color as described in the table below (color references are to The Royal Horticultural Society Colour Chart, 4th edition):

TABLE 2
Comparison with Similar Variety

| Characteristic | ‘SAKIMP023’ | ‘SAKIMP013’ |
|---|---|---|
| Petal color, upper | RHS 65B (Red-Purple) with | RHS 65C (Red-Purple) |
| surface | RHS 65A (Red-Purple) at | |
| | midvein | |
| Petal color, lower | RHS 65C (Red-Purple) with | RHS 65C (Red-Purple) |
| surface | RHS 65B (Red-Purple) at | |
| | midvein | |
| Petal color, eye | RHS N66A (Red-Purple) and | RHS 67C (Red-Purple) |
| zone | RHS 145D (Yellow-Green) | |
| Plant growth habit | Vigorous | Compact |

We claim:

1. A new and distinct cultivar of New Guinea Impatiens hybrid plant as shown and described herein.
